(19) United States
(12) Patent Application Publication
(10) Pub. No.: US 2025/0127976 A1
(43) Pub. Date: Apr. 24, 2025

(54) FILTER DEVICE FOR HUMAN BLOOD REINFUSION

(71) Applicant: INNOVA VASCULAR, INC., Irvine, CA (US)

(72) Inventors: Greg Bak-Boychuk, Irvine, CA (US); Jake Le, Irvine, CA (US); Sanjay Shrivastava, Irvine, CA (US); Cheng Ian, Irvine, CA (US)

(21) Appl. No.: 18/921,500

(22) Filed: Oct. 21, 2024

Related U.S. Application Data
(60) Provisional application No. 63/545,009, filed on Oct. 20, 2023.

(51) Int. Cl. A61M 1/36; A61M 1/34

(52) U.S. Cl. CPC A61M 1/3635; A61M 1/34

(57) ABSTRACT

The present invention features a filter canister device for filtering clot material from whole blood. The device includes an upper filter chamber and a lower filter chamber coupled to the upper filter chamber. The device further includes a coarse filter mesh disposed within the upper filter chamber, configured to capture the clot material from whole blood directed into the upper filter chamber, and a fine filter mesh disposed within the lower filter chamber, configured to filter the whole blood into filtered whole blood. The device further includes an inlet port fluidly coupled to the upper filter chamber, configured to accept the whole blood into the upper filter chamber. The device further includes an outlet port fluidly coupled to the lower filter chamber, configured to accept the filtered whole blood from the lower filter chamber and direct it to an external source.

### CROSS-REFERENCES TO RELATED APPLICATIONS

This application is a non-provisional and claims benefit of U.S. Provisional Application No. 63/545,009 filed Oct. 20, 2023, the specification of which is incorporated herein in its entirety by reference.

### FIELD OF THE INVENTION

The present invention is directed to medical devices for use in thrombectomy procedures, specifically, blood filtration canisters for filtering clot material from whole blood for reinfusion into the body.

### BACKGROUND OF THE INVENTION

Surgical procedures involving intervention into human organs will typically lead to blood loss due to the procedural steps involved in the medical intervention. Specifically “interventional procedures” where access to human organs via the human vascular system may lead to the loss of human blood. A typical adult human's vascular system will have a volume of approximately 5 liters. Minor surgical interventions where a few milliliters of whole blood is spared will not have a detrimental impact on the overall profusion of blood to the organs, and therefore will not pose a significant health risk to the patient and will not impact the healing process.

More significant interventional procedures may result in significantly larger volumes of blood loss. Blood loss greater than 250 mL can impact the overall profusion of the vascular system which can have negative health consequences, especially in humans in compromised health situations. It is therefore beneficial to either limit the blood loss associated with the intervention or in some cases have the ability to reinfuse the whole blood back into the patient.

One such medical procedure that relies upon the evacuation of relatively large volumes of blood from a patient's vascular system is called a thrombectomy. In this type of procedure, a catheter is introduced directly into the human vasculature system with the intended goal of removing blood clots from the vascular system. These clots can form in various places within either the venous circulatory system or the arterial circulatory system. Blood clots can restrict the flow of oxygenated blood to target organs, it is therefore important to remove these clots to restore optimal profusion.

The very basic steps of this type of procedure can be outlined as follows: 1) gain access to the vascular system, 2) navigate a flexible catheter shaft to the target vessel location (typically guided by X-ray fluoroscopy), and 3) aspirate (apply vacuum suction) or mechanically dislodge and retrieve the clot from the vessel. The sequence described above relies upon either aspiration or mechanical retrieval of blood from the vessel. The volume and rate of aspiration need to be higher than the blood flow so that the velocity pressure induced by aspiration will dislodge the clot material. Typically, the volume to aspirate the clot into the aspiration syringe can be as high as 100 c per aspiration attempt. Often multiple aspiration attempts are needed to clear the clot from the vessel. It is not uncommon for total volumes to exceed 350 mL.

Due to the relative volume of blood withdrawn to clear the vessel of the clot, it is desirable to reinfuse viable whole blood into the patient to limit blood loss. The aspired blood collected in the syringe will be mixed with clot material, so simply reinfusing this material is not advised. The need for a blood filtration device where the whole blood and clot materials could be separated would allow for the reinfusion of filtered blood into the patient, thus conserving the amount of blood loss associated with this type of interventional procedure. It is important to note that the operating clinician is mainly focused on clot removal, visualization (X-ray), patient physiology, patient wellbeing, and minimizing procedure time. It is therefore very important for the filtration device to be easy to use.

Key features of an easy-to-use blood filtration device are summarized as follows: 1) Stable table-top, or handheld ridged design configuration of the filtration system. The filtration device shall remain stable as the clinician debrides the aspirated material into the filter device. 2) It is preferable if, when evacuating the aspiration syringe into the filter device, the clinician is able to do so with only one hand operation (note that the operation clinician is often managing the vascular access devices at the same time), or if both hands are required to evacuate the aspiration device and simultaneously drawing the filtered blood into the reinfusion syringe that the device is rigid and stable. 3) The ability to debride the aspiration syringe quickly so that additional aspiration attempts can be conducted in quick succession (often several aspirations are required to remove the entire clot mass).

4) In the case of a vented tabletop filtration device, the ability for a technician, or fellow clinician, to withdraw filtered whole blood via a standard syringe and to reinfuse the filtered whole blood back into the patient without the need to distract the operating clinician. 5) In the case of a tabletop filtration device, a vented system so that both the aspiration syringe and the reinfusion syringe don't have to be operated simultaneously in order to simply evacuate the aspiration system. 6) The ability for the clinician, or the clinical technician, to easily open the filtration device to examine captured clot material. This information is very helpful for the operating clinician as he/she assesses if they have removed all the clot material. 7) The need to quickly assess the clot volume 8) The ability to easily clean the filtration device for subsequent filtration cycles. 9) a large filtration surface area to prevent foaming of the blood This is typically done by flushing with sterile saline.

Commercially available devices provide adequate filtration but are awkward to handle and difficult to use. They require the operating clinician to interrupt the procedure to debride the aspiration syringe while at the same time aspirating the reinfusion syringe via what is called a “push and pull” technique. There is no way to assess how much clot was aspirated until after the awkward filtration steps. This push and pull technique requires that the operating clinician uses both hands simultaneously and it is therefore very important for the device design to be easy to handle for an average operator. Also, the pressurization and vacuum pressures associated with a pressurized device can result in the foaming of the blood.

### BRIEF SUMMARY OF THE INVENTION

It is an objective of the present invention to provide devices and methods that allow for the filtration of clot material from whole blood for reinfusion during thrombectomy procedures, as specified in the independent claims. Embodiments of the invention are given in the dependent claims. Embodiments of the present invention can be freely combined with each other if they are not mutually exclusive.

The present invention features a filter canister device for filtering clot material from whole blood. The device may comprise an upper filter chamber and a lower filter chamber removably attached and fluidly coupled to the upper filter chamber. The device may further comprise a coarse filter mesh disposed within the upper filter chamber, configured to capture the clot material from whole blood directed into the upper filter chamber. The device may further comprise a fine filter mesh disposed within the lower filter chamber, configured to filter the whole blood into filtered whole blood. The device may further comprise an inlet port fluidly coupled to the upper filter chamber, configured to accept the whole blood into the upper filter chamber. The device may further comprise an outlet port fluidly coupled to the lower filter chamber, configured to accept the filtered whole blood from the lower filter chamber and direct it to an external source. The device comprises only two filter mesh layers.

One of the unique and inventive technical features of the present invention is the implementation of only two filter mesh layers, one finer than the other, in a filter canister device for blood filtering and clot capturing. Without wishing to limit the invention to any theory or mechanism, it is believed that the technical feature of the present invention advantageously provides for greater ease of assembly/disassembly of the device for quickly analyzing captured material while still achieving efficient removal of clot material from whole blood without damage to blood cells or reactivation of clot material. None of the presently known prior references or work has the unique inventive technical feature of the present invention.

Furthermore, the inventive technical features of the present invention contributed to a surprising result. Prior systems have implemented more than two filter meshes of differing porosities in order to achieve filtering and capture of materials found in aspirated blood. Surprisingly, the present invention is able to achieve efficient and safe filtering and reinfusion of whole blood using only two layers of filtering, while avoiding the reactivation of clot material and damaging red blood cells. Thus the inventive technical features of the present invention contributed to a surprising result.

Another one of the unique and inventive technical features of the present invention is the ability to visualize clot material as captured by the filter mesh immediately upon dispensing the whole blood into the filter canister. Without wishing to limit the invention to any theory or mechanism, it is believed that the technical feature of the present invention advantageously provides for greater time efficiency in clot aspiration and blood reinfusion procedures. Instead of needing to extract the blood from the canister to know if clot material was captured, the present invention allows the medical professional executing the procedure to know if clot material was extracted instantly. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a diagram of an embodiment of the blood filtration device of the present invention comprising an angled inlet port and a plurality of base legs configured to rest on a leg of a patient.

FIG. 1B shows a diagram of an alternate embodiment of the blood filtration system of the present invention.

FIG. 1C shows a flow chart of a method for blood filtration of the present invention.

FIG. 2A shows a diagram of a syringe directing whole blood through the inlet of the blood filtration device of the present invention.

FIG. 2B shows a diagram of a syringe extracting filtered blood through the outlet of the blood filtration device of the present invention.

FIG. 3 shows an exploded view of the blood filtration device of the present invention, showing captured clot material.

FIG. 4 shows an exploded view of the blood filtration device of the present invention.

FIG. 5A shows a diagram of the blood filtration device of the present invention in an embodiment comprising a ball valve.

FIG. 5B shows an interior view of the ball valve of the present invention while the buoyant component is blocking the air vent.

FIG. 5C shows an interior view of the ball valve of the present invention while the buoyant component is not blocking the air vent.

FIG. 5D shows the embodiment of the blood filtration device of the present invention comprising the ball valve in use.

FIG. 5E shows a process of extracting filtered blood from the blood filtration device of the present invention comprising a ball valve while clot material remains.

FIG. 6A shows an alternate embodiment of the blood filtration device of the present invention comprising a check valve.

FIG. 6B shows an alternate embodiment of the blood filtration device of the present invention comprising a check valve, containing captured clot material.

FIG. 6C shows an exploded view of the alternate embodiment of the blood filtration device of the present invention comprising a check valve.

FIG. 7A shows an interior view of the check valve of the present invention preventing air flow.

FIG. 7B shows an interior view of the check valve of the present invention allowing air flow.

FIG. 7C shows an exploded view of the check valve of the present invention as it is implemented into the upper filter chamber.

FIG. 8A shows an alternate handheld embodiment of the blood filtration device of the present invention while in use.

FIG. 8B shows an exploded view of the handheld embodiment of the blood filtration device of the present invention.

FIG. 9 shows an interior closeup view of the upper filter chamber and lower filter chamber of the blood filtration device of the present invention.

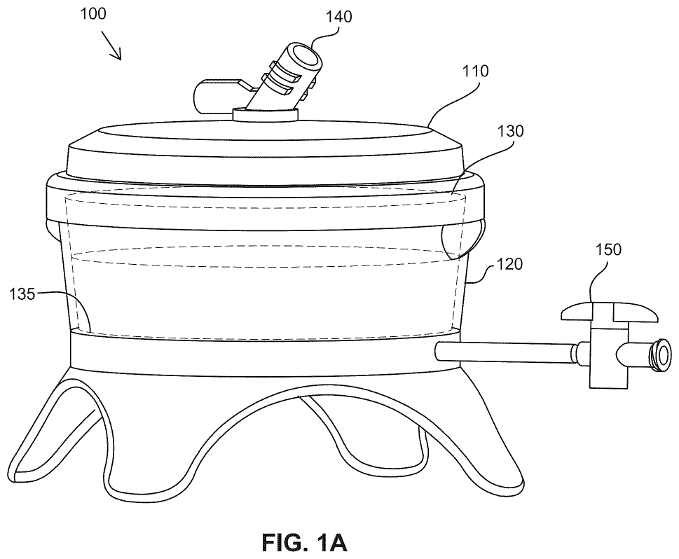

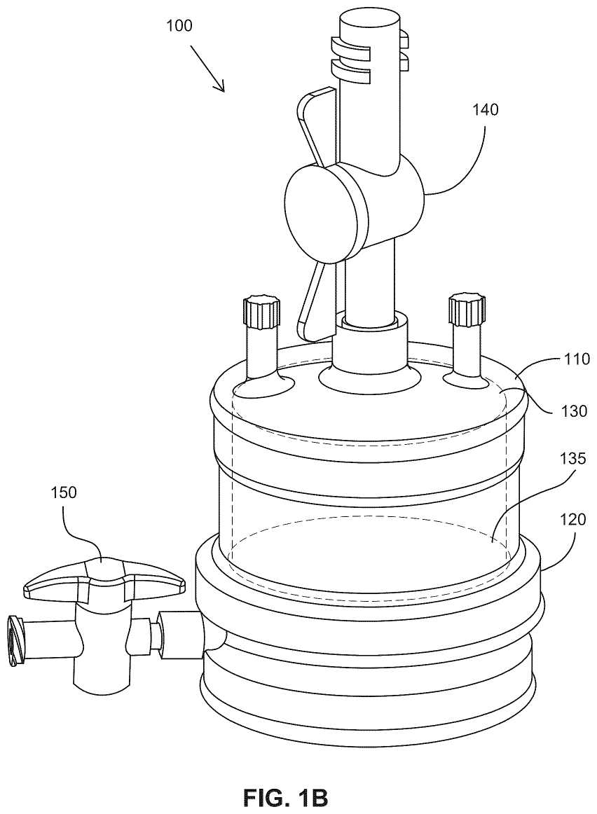

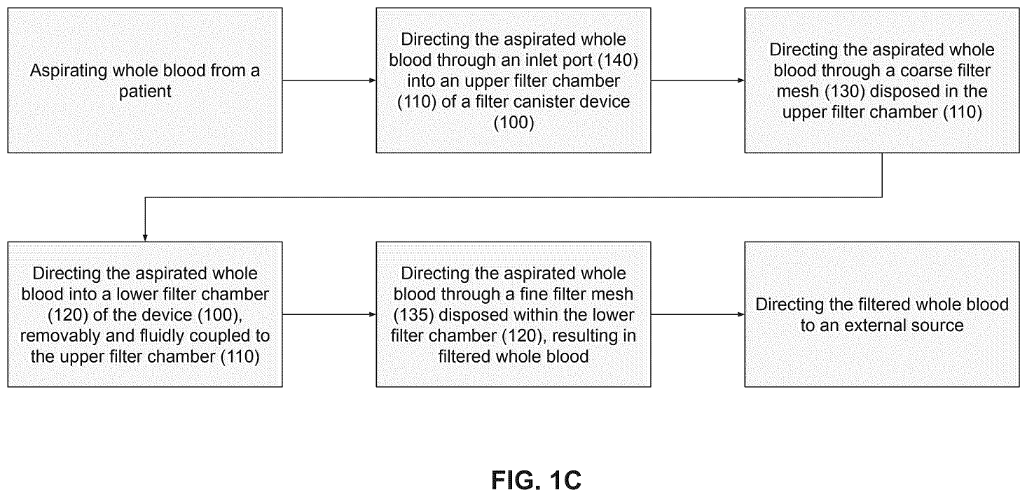

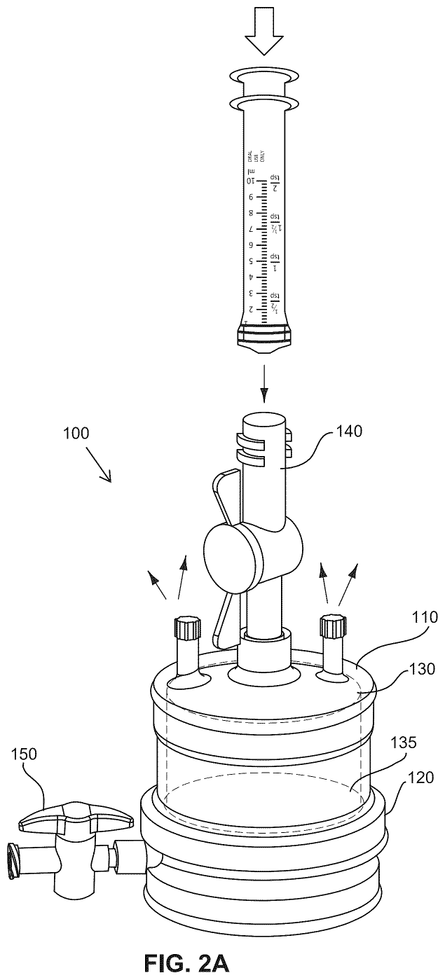

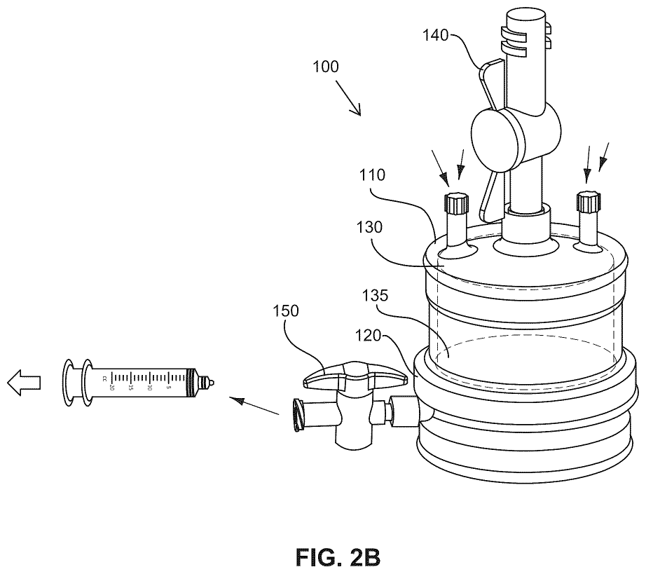

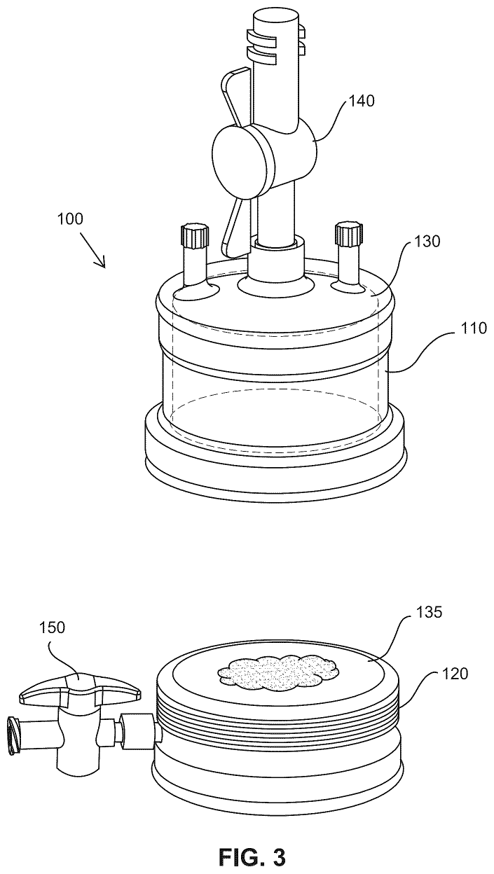

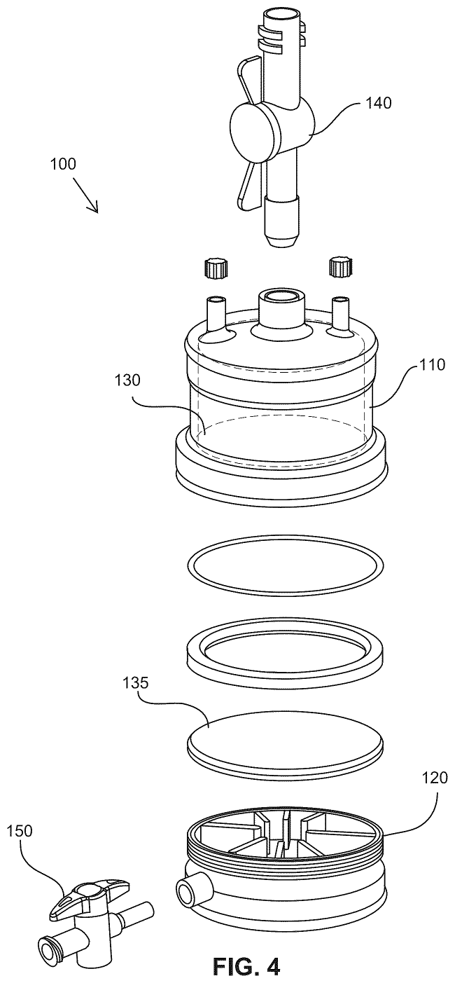

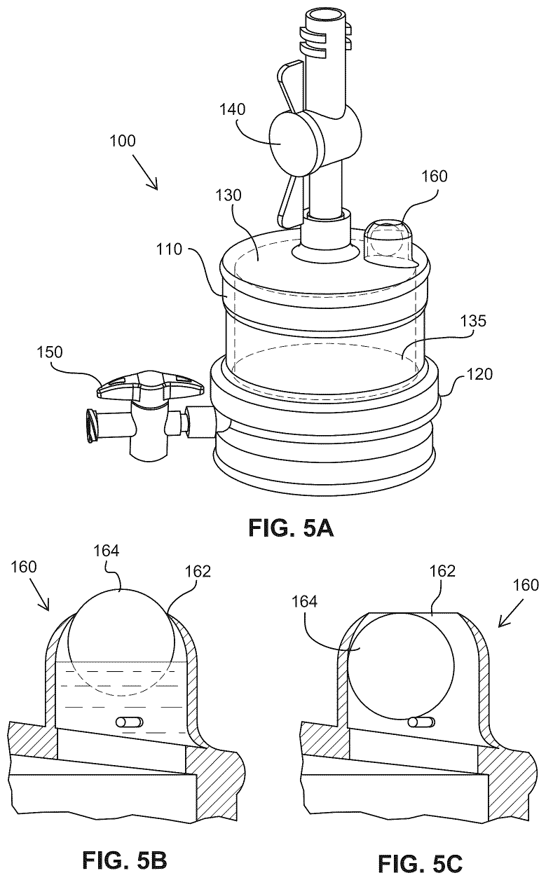

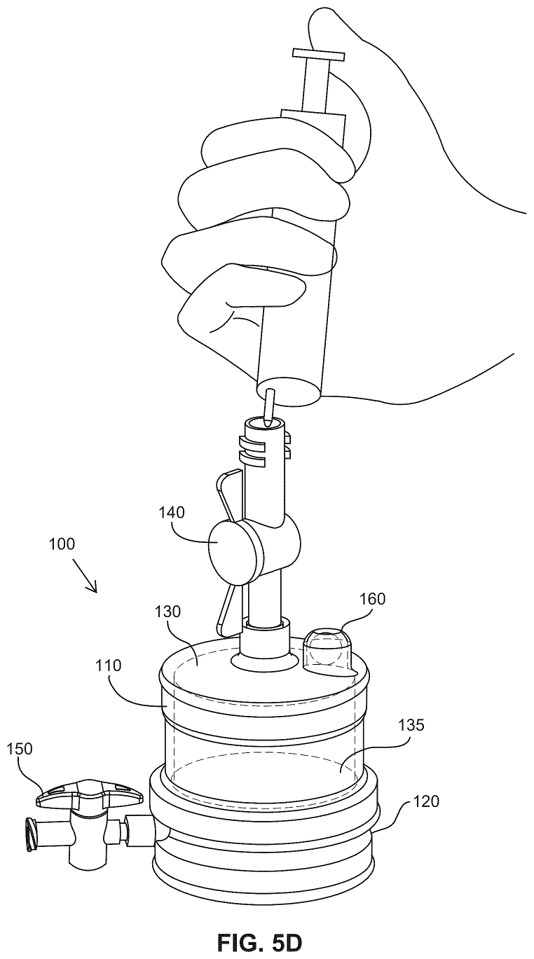

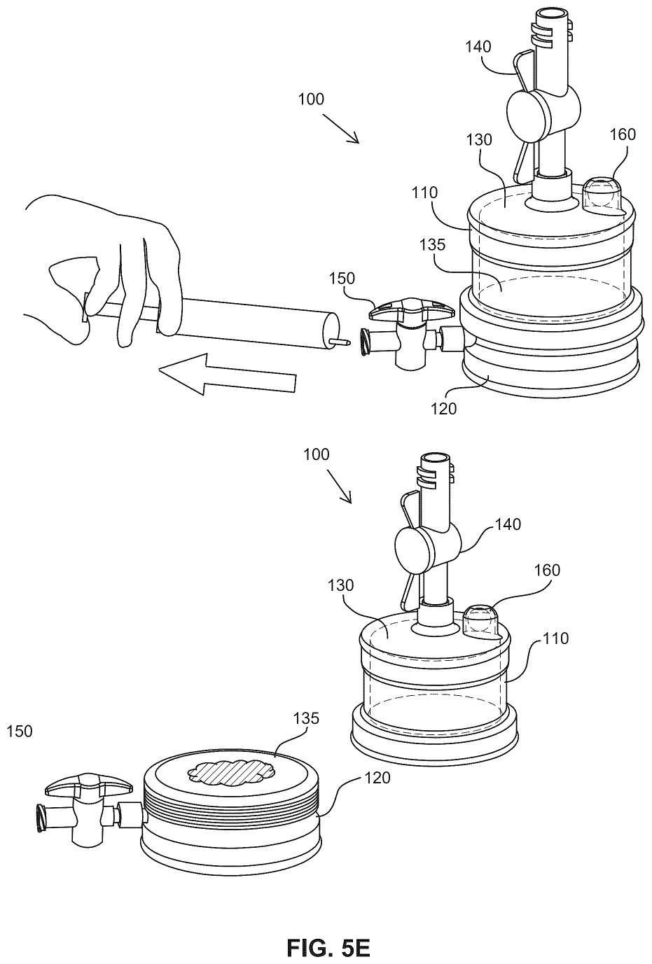

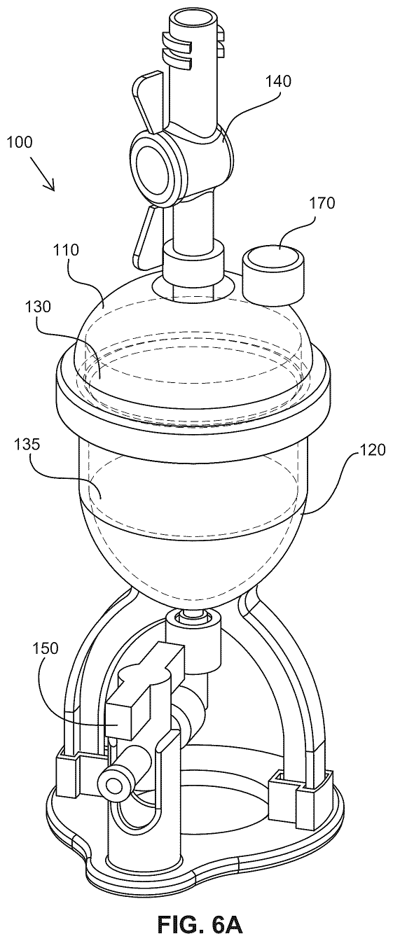

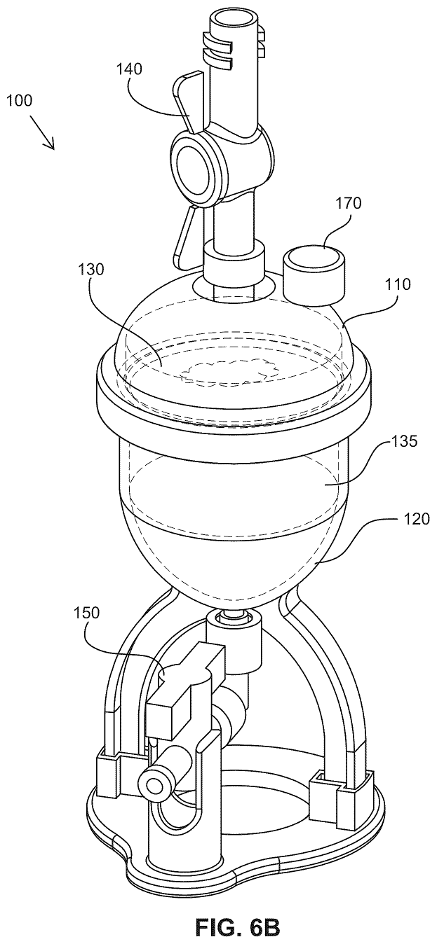

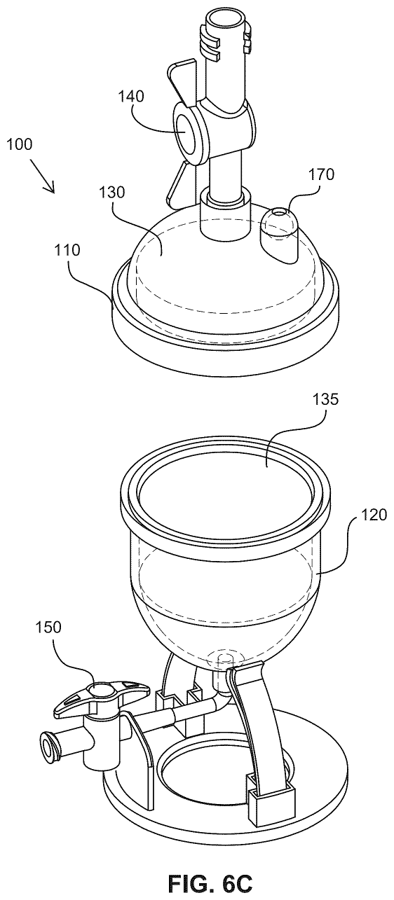

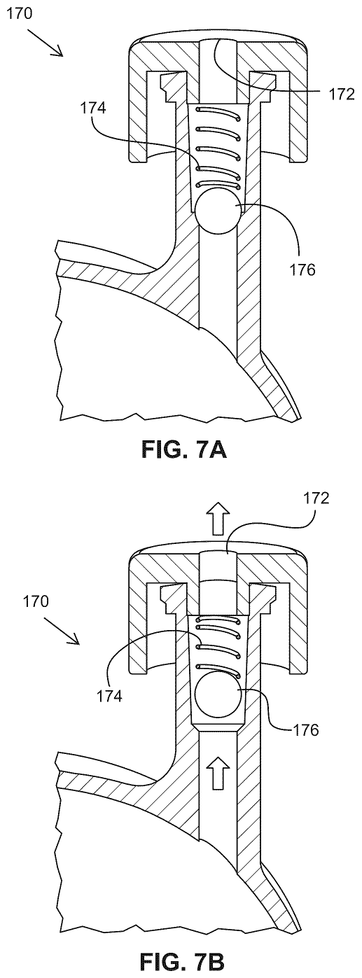

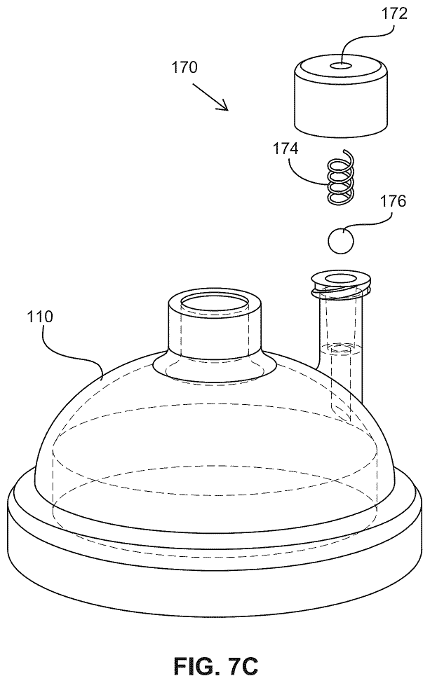

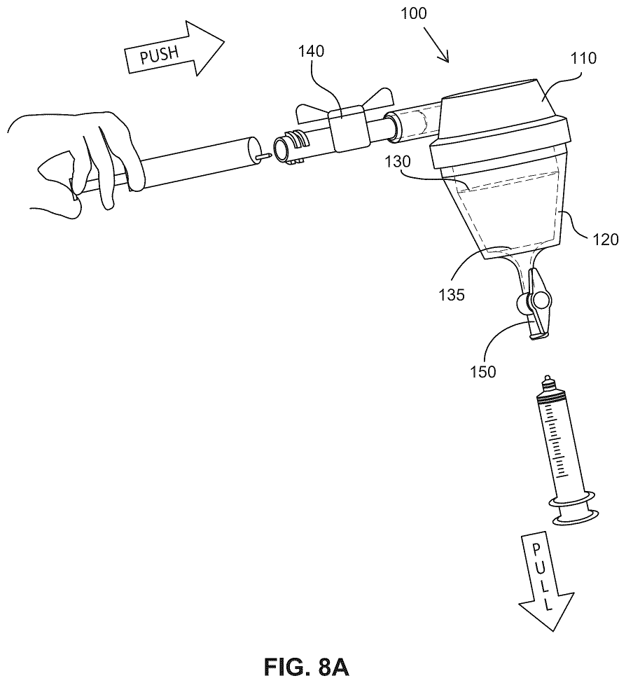

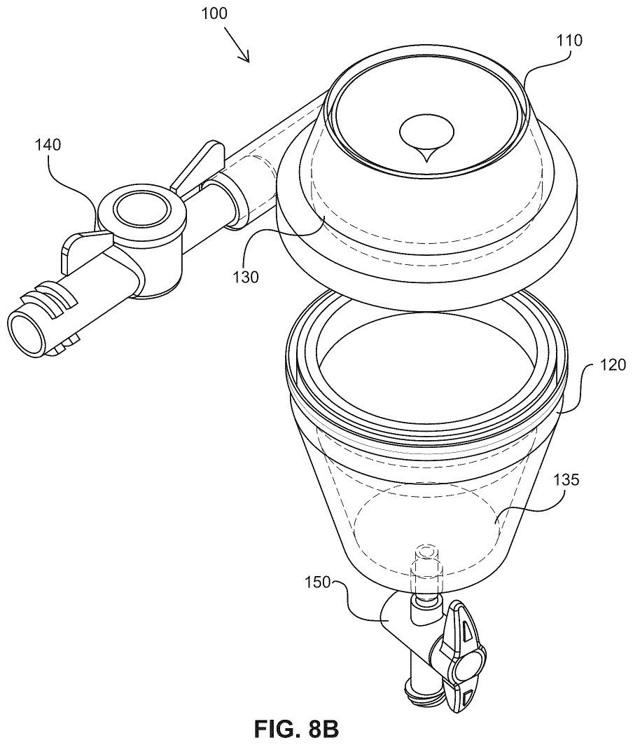

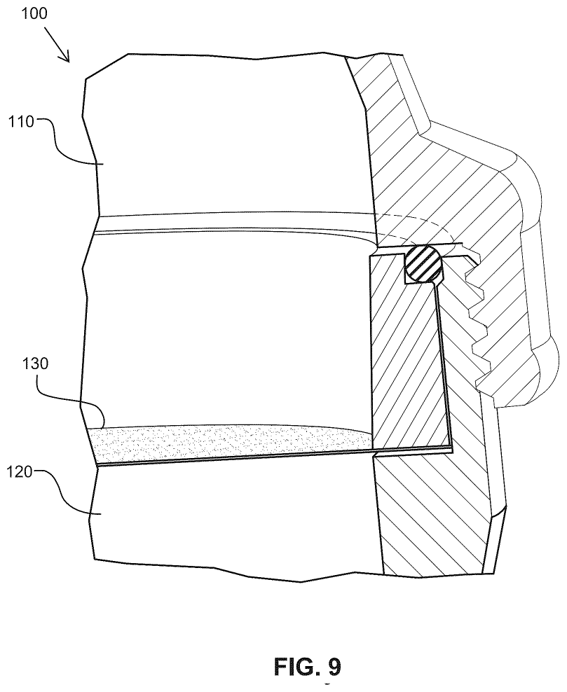

### DETAILED DESCRIPTION OF THE INVENTION

Following is a list of elements corresponding to a particular element referred to herein:
100 device
110 upper filter chamber
120 lower filter chamber
130 coarse filter mesh
135 fine filter mesh
140 inlet port
150 outlet port
160 float vent
162 first air vent
164 buoyant component
170 check valve
172 second air vent
174 spring
176 blocking component

The term “whole blood” is defined herein as blood directly extracted from a body, containing red blood cells, white blood cells, platelets, and blood plasma.

The term “coarse” is defined herein as a filter mesh having a porosity greater than or equal to 1000 microns.

The term “fine” is defined herein as a filter mesh having a porosity less than or equal to 100 microns.

The term “float vent” is defined herein as an air vent comprising a component configured to prevent air flow when it is floating and allow air flow when it is not.

The present invention features a filter canister device (100) for filtering clot material from whole blood. In some embodiments, the device (100) may comprise an upper filter chamber (110), configured to receive and contain aspirated whole blood. The upper filter chamber (110) may be at least partially transparent. The device (100) may further comprise a lower filter chamber (120) removably attached and fluidly coupled to the upper filter chamber (110), configured to receive and contain filtered whole blood.

The device (100) may further comprise a coarse filter mesh (130) disposed within the upper filter chamber (110), configured to capture the clot material from the aspirated whole blood. The device (100) may further comprise a fine filter mesh (135) disposed within the lower filter chamber (120), configured to filter the aspirated whole blood into filtered whole blood. The device (100) may further comprise an inlet port (140) fluidly coupled to the upper filter chamber (110), configured to accept the aspirated whole blood and direct the aspirated whole blood into the upper filter chamber (110). The device (100) may further comprise an outlet port (150) fluidly coupled to the lower filter chamber (120), configured to accept the filtered whole blood from the lower filter chamber (120) and direct the filtered whole blood to an external source. The device (100) comprises only two filter mesh layers.

In some embodiments, the inlet port (140) may comprise a stopcock valve. In some embodiments, the outlet port (150) may comprise a stopcock valve. In some embodiments, the upper filter chamber (110) and the lower filter chamber (120) may be non-vented. In some embodiments, the device (100) may further comprise one or more air vents fluidly coupled to the upper filter chamber (110). In some embodiments, the one or more air vents may comprise one or more float vents. Each float vent (160) may comprise an air port (162) fluidly coupled to air surrounding the device (100) such that air flow is provided in the upper filter chamber (110). Each float vent (160) may further comprise a buoyant component (164) disposed in-line with the air port (162), configured to rise with a fluid level within the upper filter chamber (110) such that the buoyant component (164) blocks the air port (162) at a maximum fluid level in the upper filter chamber (110).

In some embodiments, the one or more air vents may comprise one or more check valves. Each check valve (170) may comprise an air port (172) fluidly coupled to air surrounding the device (100) such that air flow is provided in the upper filter chamber (110). Each check valve (170) may further comprise a spring (174) disposed in-line with the air port (172), configured to move from an extended configuration to a compressed configuration when pressure is applied and move from the compressed configuration to the extended configuration when the pressure is released. Each check valve (170) may further comprise a blocking component (176) coupled to the spring (174) such that the blocking component (176) is configured to block the air port (172) when the spring (174) is in the extended configuration, and the blocking component (176) is configured to release from the air port (172) when the spring (174) is in the compressed configuration such that air flow is permitted through the air port (172). The aspirated whole blood, upon being directed into the upper filter chamber (110), may generate the pressure in the upper filter chamber (110) such that the pressure is applied to the spring (174). The filtered whole blood, upon being directed out of the lower filter chamber (120), may release the pressure in the upper filter chamber (110) such that the pressure is released from the spring (174).

In some embodiments, the device (100) may further comprise a plurality of base legs disposed on the lower filter chamber (120). The plurality of base legs may be configured to support the device (100) on a leg of a patient. In some embodiments, the inlet port (140) may be configured to accept a syringe, a catheter, or a combination thereof. In some embodiments, the outlet port (150) may be configured to accept a syringe, a catheter, or a combination thereof.

In some embodiments, the present invention features a filter canister device (100) for filtering clot material from whole blood. In some embodiments, the device (100) may comprise an upper filter chamber (110), configured to receive and contain aspirated whole blood. The upper filter chamber (110) may be at least partially transparent. The device (100) may further comprise a lower filter chamber (120) removably attached and fluidly coupled to the upper filter chamber (110), configured to receive and contain filtered whole blood. The device (100) may further comprise a coarse filter mesh (130) disposed within the upper filter chamber (110), configured to capture the clot material from the aspirated whole blood. The device (100) may further comprise a fine filter mesh (135) disposed within the lower filter chamber (120), configured to filter the aspirated whole blood into filtered whole blood.

The device (100) may further comprise an inlet port (140) fluidly coupled to the upper filter chamber (110), configured to accept the aspirated whole blood and direct the aspirated whole blood into the upper filter chamber (110). The device (100) may further comprise an outlet port (150) fluidly coupled to the lower filter chamber (120), configured to accept the filtered whole blood from the lower filter chamber (120) and direct the filtered whole blood to an external source.

The device (100) may further comprise one or more float vents fluidly coupled to the upper filter chamber (110). Each float vent (160) may comprise a first air port (162) fluidly coupled to air surrounding the device (100) such that air flow is provided in the upper filter chamber (110). Each float vent (160) may further comprise a buoyant component (164) disposed in-line with the first air port (162), configured to rise with a fluid level within the upper filter chamber (110) such that the buoyant component (164) blocks the first air port (162) at a maximum fluid level in the upper filter chamber (110). The device (10)) may further comprise one or more check valves fluidly coupled to the upper filter chamber (110), each check valve (170) may comprise a second air port (172) fluidly coupled to air surrounding the device (100) such that air flow is provided in the upper filter chamber (110). Each check valve (170) may further comprise a spring disposed in-line with the second air port (172), configured to move from an extended configuration to a compressed configuration when pressure is applied and move from the compressed configuration to the extended configuration when the pressure is released. Each check valve (170) may further comprise a blocking component (176) coupled to the spring (174) such that the blocking component (176) is configured to block the second air port (172) when the spring (174) is in the extended configuration, and the blocking component (176) is configured to release from the second air port (172) when the spring (174) is in the compressed configuration such that air flow is permitted through the second air port (172).

The aspirated whole blood, upon being directed into the upper filter chamber (110), may generate the pressure in the upper filter chamber (110) such that the pressure is applied to the spring (174). The filtered whole blood, upon being directed out of the lower filter chamber (120), may release the pressure in the upper filter chamber (110) such that the pressure is released from the spring (174). The device (100) may comprise only two filter mesh layers.

In some embodiments, the device (100) may comprise a plurality of base legs disposed on the lower filter chamber (120). The plurality of base legs may be configured to support the device (100) on a leg of a patient. In some embodiments, the inlet port (140) may be configured to accept a syringe, a catheter, or a combination thereof. In some embodiments, the outlet port (150) may be configured to accept a syringe, a catheter, or a combination thereof. In some embodiments, the inlet port (140) may comprise a stopcock valve. In some embodiments, the outlet port (150) may comprise a stopcock valve.

The present invention features a method for filtering clot material from whole blood. In some embodiments, the method may comprise aspirating whole blood from a patient. The method may further comprise directing the aspirated whole blood through an inlet port (140) into an upper filter chamber (110) of a filter canister device (100). The method may further comprise directing the aspirated whole blood through a coarse filter mesh (130) disposed in the upper filter chamber (110) such that the coarse filter mesh (130) captures the clot material from the aspirated whole blood. The method may further comprise visualizing the clot material as captured by the coarse filter mesh (130). The method may further comprise directing the aspirated whole blood into a lower filter chamber (120) of the device (100), removably and fluidly coupled to the upper filter chamber (110). The method may further comprise directing the aspirated whole blood through a fine filter mesh (135) disposed within the lower filter chamber (120), resulting in filtered whole blood. The method may further comprise directing the filtered whole blood to an external source. The device (100) may comprise only two filter mesh layers.

In some embodiments, the present invention features a method for preventing foam buildup while filtering clot material from whole blood. In some embodiments, the method may comprise aspirating whole blood from a patient, directing the aspirated whole blood through an inlet port (140) into an upper filter chamber (110) of a vented, non-pressurized filter canister device (100), directing the aspirated whole blood through a coarse filter mesh (130) disposed in the upper filter chamber (110) such that the coarse filter mesh (130) captures the clot material from the aspirated whole blood, visualizing the clot material as captured by the coarse filter mesh (130), directing the aspirated whole blood into a lower filter chamber (120) of the device (100), removably and fluidly coupled to the upper filter chamber (110), directing the aspirated whole blood through a fine filter mesh (135) disposed within the lower filter chamber (120), resulting in filtered whole blood, and directing the filtered whole blood to an external source. The device (100) may comprise only two filter mesh layers.

The present invention features a kit for filtering clot material from whole blood. In some embodiments, the kit may comprise a filter canister device (100). The filter canister device may comprise an upper filter chamber (110), configured to receive and contain aspirated whole blood. The upper filter chamber (110) may be at least partially transparent. The device (100) may further comprise a lower filter chamber (120) removably attached and fluidly coupled to the upper filter chamber (110), configured to receive and contain filtered whole blood. The device (100) may further comprise a coarse filter mesh (130) disposed within the upper filter chamber (110), configured to capture the clot material from the aspirated whole blood. The device (100) may further comprise a fine filter mesh (135) disposed within the lower filter chamber (120), configured to filter the aspirated whole blood into filtered whole blood. The device (100) may further comprise an inlet port (140) fluidly coupled to the upper filter chamber (110), configured to accept the aspirated whole blood and direct the aspirated whole blood into the upper filter chamber (110). The device (100) may further comprise an outlet port (150) fluidly coupled to the lower filter chamber (120), configured to accept the filtered whole blood from the lower filter chamber (120) and direct the filtered whole blood to an external source. The device (100) comprises only two filter mesh layers.

In some embodiments, the kit may further comprise an inlet syringe configured to aspirate whole blood from the body, contain the aspirated whole blood, and direct the aspirated whole blood through the inlet port (140). In some embodiments, the kit may further comprise an inlet catheter having a first end and a second end. The first end may be configured to couple to the inlet port (140). In some embodiments, the second end may be configured to couple to the inlet syringe. In some embodiments, the second end may be configured to couple directly to the body, acting as an aspiration catheter. In some embodiments, the kit may further comprise an inlet pump fluidly coupled to the inlet catheter, configured to carry out a pumping mechanism such that the whole blood from the body is pumped through the inlet catheter and through the inlet port (140) into the upper filter chamber (110).

In some embodiments, the kit may further comprise an outlet syringe configured to couple to the outlet port (150), extract the filtered whole blood from the lower filter chamber (120), contain the filtered whole blood, and direct the filtered whole blood back into the body. In some embodiments, the kit may further comprise an outlet catheter having a first end and a second. The first end may be configured to couple to the outlet port (150). In some embodiments, the second end may be configured to couple to the outlet syringe. In some embodiments, the second end may be configured to couple directly to the body, acting as a reinfusion catheter. In some embodiments, the kit may further comprise an outlet pump fluidly coupled to the outlet catheter, configured to carry out a pumping mechanism such that the filtered whole blood is extracted from the lower filter chamber (120), through the outlet port (150), through the outlet catheter and back into the body.

In some embodiments, the kit may further comprise a saline source. The kit may further comprise a flushing syringe configured to extract saline from the saline source. The filter canister device (100) may be flushed by using the flushing syringe to direct saline through the inlet port (140), through the upper filter chamber (110), through the coarse filter mesh (130), through the fine filter mesh (135), and into the lower filter chamber (120). The flushing syringe may then be used to extract the saline from the lower filter chamber (120) through the outlet port (150). In some embodiments, the inlet syringe may be further configured to extract saline from the saline source and expel the saline to flush the inlet syringe. In some embodiments, the outlet syringe may be further configured to extract saline from the saline source and expel the saline to flush the outlet syringe. In some embodiments, the flushing syringe may be further configured to direct saline from the saline source through the inlet catheter, the outlet catheter, or a combination thereof.

In some embodiments, the inlet port (140) may comprise a stopcock valve. In some embodiments, the outlet port (150) may comprise a stopcock valve. In some embodiments, the upper filter chamber (110) and the lower filter chamber (120) may be non-vented. In some embodiments, the device (100) may further comprise one or more air vents fluidly coupled to the upper filter chamber (110). In some embodiments, the one or more air vents may comprise one or more float vents. Each float vent (160) may comprise an air port (162) fluidly coupled to air surrounding the device (100) such that air flow is provided in the upper filter chamber (110). Each float vent (160) may further comprise a buoyant component (164) disposed in-line with the air port (162), configured to rise with a fluid level within the upper filter chamber (110) such that the buoyant component (164) blocks the air port (162) at a maximum fluid level in the upper filter chamber (110).

In some embodiments, the one or more air vents may comprise one or more check valves. Each check valve (170) may comprise an air port (172) fluidly coupled to air surrounding the device (100) such that air flow is provided in the upper filter chamber (110). Each check valve (170) may further comprise a spring (174) disposed in-line with the air port (172), configured to move from an extended configuration to a compressed configuration when pressure is applied and move from the compressed configuration to the extended configuration when the pressure is released. Each check valve (170) may further comprise a blocking component (176) coupled to the spring (174) such that the blocking component (176) is configured to block the air port (172) when the spring (174) is in the extended configuration, and the blocking component (176) is configured to release from the air port (172) when the spring (174) is in the compressed configuration such that air flow is permitted through the air port (172). The aspirated whole blood, upon being directed into the upper filter chamber (110), may generate the pressure in the upper filter chamber (110) such that the pressure is applied to the spring (174). The filtered whole blood, upon being directed out of the lower filter chamber (120), may release the pressure in the upper filter chamber (110) such that the pressure is released from the spring (174).

In some embodiments, the device (100) may further comprise a plurality of base legs disposed on the lower filter chamber (120). The plurality of base legs may be configured to support the device (100) on a leg of a patient. In some embodiments, the inlet port (140) may be configured to accept a syringe, a catheter, or a combination thereof. In some embodiments, the outlet port (150) may be configured to accept a syringe, a catheter, or a combination thereof.

In some embodiments, the device (100) may comprise a plurality of base legs disposed on the lower filter chamber (120). The method may further comprise supporting the device (100) on a leg of the patient. In some embodiments, the inlet port (140) may be configured to accept a syringe, a catheter, or a combination thereof. In some embodiments, the outlet port (150) may be configured to accept a syringe, a catheter, or a combination thereof.

In some embodiments, the buoyant component (164) may comprise a block. In some embodiments, the buoyant component (164) may comprise a ball. In some embodiments, the buoyant component (164) may comprise any solid or semi-solid component comprising a buoyant material and shaped to cover the air port (162) and prevent airflow. The float vent (160) may comprise a chamber having a diameter greater than the air port (162) such that while the buoyant component (164) is not floating, the buoyant component (164) rests below the air port and air is able to freely flow. In some embodiments, the float vent may further comprise a grooved platform configured to hold the buoyant component (162) while it is not floating. In some embodiments, the float vent may be shaped such that as the chamber fills with fluid, the buoyant component (162) is directed towards the air port (162) for blocking.

In some embodiments, the blocking component (176) may comprise a block. In some embodiments, the blocking component (176) may comprise a ball. In some embodiments, the blocking component (176) may comprise any solid or semi-solid component shaped to cover the air port (172). In some embodiments, the check valve (170) may comprise a chamber having a diameter greater than the air port (172) such that while the spring (174) is in the compressed configuration and the blocking component (176) is not blocking the air port (172), the air is able to flow freely. In some embodiments, the blocking component (176) is configured to rest on the spring (174). In some embodiments, the spring (174) is aligned with the air port (172) such that the blocking component (176) aligns with the air port (172) while the spring (174) is in the extended configuration.

In some embodiments, the blocking component (176) may be configured to block the air port (172) while the spring (174) is in the compressed configuration. For example, the chamber may comprise a decreased diameter near a bottom portion of the spring (174) such that the blocking component (176) plugs up the chamber while the spring (174) is in the compressed configuration. Conversely, the chamber may comprise a greater diameter at an upper portion of the spring (174) while the spring is in the extended configuration such that the chamber is unplugged and air can freely flow through the air port (172) into the upper filter chamber (110).

In some embodiments, the blocking component (176) may be configured to block the air port (172) while the spring is in the extended configuration. For example, the spring (174) may be aligned with the air port (172) such that when the spring (174) is in the extended configuration, the blocking component (176) is pushed up to block the air port (172). Conversely, when the spring (174) is in the compressed configuration, the blocking component (176) is pulled away from the air port (172), thus allowing air to freely flow through the air port (172) into the upper filter chamber (110).

In some embodiments, a porosity of the coarse filter mesh (130) may be 700 to 1500 microns. In some embodiments, a porosity of the fine filter mesh (135) may be 40 to 100 microns. In some embodiments, the upper filter chamber (110) may comprise plastic, glass, any other solid and transparent material, or a combination thereof. In some embodiments, the lower filter chamber (120) may comprise plastic, glass, metal, any other solid material suitable for interaction with the material of the upper filter chamber (110), or a combination thereof.

In some embodiments, the coarse filter mesh (130) may comprise woven wire mesh (stainless steel). In some embodiments, the fine filter mesh (135) may comprise woven wire mesh (stainless steel). In some embodiments, the coarse filter mesh (130), the fine filter mesh (135), or a combination thereof may be generated by drilling many small holes into a metal or plastic film. In some embodiments, the coarse filter mesh (130) may additionally be layered with an additional woven wire mesh (stainless steel). In some embodiments, the fine filter mesh (135) may be additionally layered with a heparin coating, a hydrophobic coating, or a combination thereof.

In some embodiments, the upper filter chamber (110) and the lower filter chamber (120) may be threaded such that the chambers are able to be easily attached and detached from each other. In some embodiments, the upper filter chamber (110) and the lower filter chamber (120) may have a snapping and locking mechanism such that the chambers are able to be easily attached and detached from each other. In some embodiments, the upper filter chamber (110) and the lower filter chamber (120) may have any mechanism binding them to each other such that a user is able to detach the chambers from each other easily with one hand.

In some embodiments, all components of the device may be easily detachable from each other in some form or fashion. The inlet port (140) may be disposed on a lid that can be detached from the upper filter chamber (110). The coarse filter mesh (130) may be removable from the upper filter chamber (130) for cleaning, visualization, or replacement. The fine filter mesh (135) may be removable from the lower filter chamber (135) for cleaning, visualization, or replacement. The outlet port (150) may be disposed on a base portion comprising the one or more base legs that may be removable from the lower filter chamber (120).

In some embodiments, the fine filter mesh (135) may comprise a filter area of 60 to 65 cm². The fine filter mesh (135) may comprise an open filter area of 20 to 25 cm². Thus, when interacting with a 60 cc syringe drawing or dispensing blood in 5 seconds, the flow rate through the fine filter mesh (135) is 12 cc/sec. This means that the shear velocity across the fine filter mesh (135) is approximately 0.48 cm/sec. This is compared to prior systems where the shear velocity across filter meshes is approximately 2.4 cm/sec. This filter area prevents damage to the red blood cells by reducing the shear velocity across the filter mesh.

In some embodiments, the coarse filter mesh (130), the fine filter mesh (135), or a combination thereof may comprise a wide, even mesh with no regions of blockage. In some embodiments, the coarse filter mesh (130) may have a percent-porosity of 40 to 60 percent. In some embodiments, the fine filter mesh (135) may have a percent porosity of 22 to 32 percent.

In some embodiments, the vented blood filtration system of the present invention may prevent foaming of the blood. Prior push-pull design filtration systems that implement pressurized chambers create a high vacuum which results in foaming when drawing filtered blood. This causes the amount of usable blood to be greatly reduced. The implementation of a vented system greatly reduces and/or prevents the buildup of foam while extracting filtered blood. In some embodiments, the vents may comprise a plurality of slits between the coarse filter mesh (130) and the upper filter chamber (110), exposing the upper filter chamber (110) to the sterile environment and preventing pressure buildup.

In some embodiments, the total volume of the lower filter chamber (120) of the present invention may be at least 200 cc. Because of this, the lower filter chamber (120) of the present invention is able to contain multiple full syringes worth of aspirated whole blood. This allows the medical official aspirating the blood to dispense multiple syringes worth of blood into the device at a time without having to stop and draw out blood every time.

In some embodiments, the shape of the lower filter chamber (120) below the fine filter mesh (135) is intended to minimize the volume below the fine filter material. In some embodiments, this volume below the fine filter mesh (135) may be less than or equal to 10 ccs. In some embodiments, this volume below the fine filter mesh (135) may be 6 to 8 ccs. This may help to filter as much blood volume as possible. If you simply “suck” or draw blood across the filter, once there is no blood on the top of the filter air will be drawn into the syringe and “filtering” stops. The fine filter mesh (135) of the present invention allows the blood to move uniformly across the surface of the filter to prevent this situation. The fine filter mesh (135) and the volume below were designed and optimized to suspend the filter element from collapse, implement a sloped bottom to draw as much blood as possible and limit traps of blood, be easy to clean, and have minimal volume.

In some embodiments, the inlet port (140) may be coupled to a pump device configured to pump the whole blood directly from the patient's body through a catheter. In some embodiments, the inlet port (140) may be configured to accept a syringe containing the whole blood such that the whole blood from the syringe can be sterilely transmitted from the syringe through the inlet port (140). In some embodiments, the inlet port (140) may be configured to accept one end of a catheter and the other end may be configured to accept a syringe containing the whole blood.

In some embodiments, the outlet port (150) may be coupled to a pump device configured to pump the filtered blood directly from the lower filter chamber (120) to the patient's body through a catheter. In some embodiments, the outer port (150) may be configured to accept a syringe configured to sterilely extract the filtered blood from the lower filter chamber (120) through the outlet port (150). In some embodiments, the outlet port (150) may be configured to accept one end of a catheter and the other end may be configured to accept a syringe configured to extract the filtered blood.

Generally, the filtration device may be constructed of plastic materials. The filtration device may be considered a single-procedure disposable device and discarded at the conclusion of the medical intervention. The device may be sterilized and packaged in a pouch to maintain a barrier to sterility. The device may incorporate a filtration media. This filter media is typically in the form of a woven mesh material. To prevent damage to blood cells, a single-layer polymer mesh, constructed of monofilament fibers spaced at 40 microns may allow the passage of 5 to 8-micron blood cells with minimal damage to the whole blood cells. Multiple polymers may be used (nylon, Teflon, polyethylene, etc.). To prevent additional clotting, a heparin coating may be added to the filter mesh material. It is best to use filter mesh materials that are smooth so as not to damage blood cells as damaged blood cells may activate further clotting.

The filtration canister may be easy to separate for clot material inspection and cleaning, and the canister may be able to withstand the pressure from the aspiration syringe (up to 30 psi). A vented canister may allow for quick single-handed debridement of the aspiration syringe. This may be accomplished by way of a vent cap, which is just a cap with a small orifice. The vent may be more complex to prevent the inadvertent overfilling of the filtration canister. The present invention may comprise a float vent to prevent overfilling. As the blood is extracted for reinfusion, the ball may drop allowing for easy filling of the reinfusion syringe. Additionally, a spring-loaded ball check valve may be incorporated into the canister. The intent of the spring-loaded ball check valve is to minimize the air circulation in the chamber after the blood materials are deposited into the canister. The ball check valve may lift due to the pressure from injecting aspirated materials into the chamber, thus venting the chamber of air. When the injection is completed, the filtered blood and the ball check valve may prevent air circulation. Air circulation is one of the contributing factors for clotting and it is therefore desirable to minimize air circulation.

The shape and design of the tabletop canister may be intended to be stable on a typical tabletop. Debridement of the aspiration syringe with one hand generates both vertical and horizontal loads on the canister body, so a broad-based and stable design may be implemented.

In the push-and-pull design, the upper chamber has a tangential port for injecting aspirated material into the filtration chamber. This tangential port, coupled with the toroidal chamber shape, is intended to decrease the velocity of the aspirated material so that the clot materials do not impact the filter element at a high velocity. This upper chamber design provides two advantages. One is that low-velocity clot materials tend to not get embedded into the filter material which makes cleaning (rinsing) the filter easier for subsequent usage. Secondly, the slower material velocity crossing the filter membrane tends to reduce the potential of blood cell clot activation (additional clotting). FIG. 15 also shows both the aspiration syringe and the syringe used to draw filtered whole blood, which are both connected to the ridged filtration device. The ridged coupling of both the inlet and decanting valves aids in the overall rigidity of the entire device and connected syringes. The 90-degree angle of the two syringes is easy to handle and manipulate by an average user. The rigidity of the overall system allows for stable and controllable push and pull operation by the user. Systems that incorporate flexible tubing between the canister and one or both syringes make the simultaneous operation of both syringes less stable and therefore more difficult for the user. The rigid 90-degree system would therefore be easier for the user to achieve the end goal of whole blood reperfusion/blood conservation.

The two chamber housings may be coupled and decoupled via a threaded connection. An o-ring may be incorporated into the lower chamber to seal the chamber halves. The depth of the clot retention pocket in the lower chamber may allow for the collection of the clot material and containment of the clot materials such that when the two chamber halves are decoupled, clot materials are contained in the device, thereby reducing the chance of debris spillage.

The filter chamber of the present invention may comprise a securing ring. The securing ring may have a tapered edge that traps the o-ring such that it does not dislodge when cleaning. The circular filter element may be secured to the lower chamber by adhesive, thermal bonding, an interference fit, by insert molding, or a combination thereof.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A filter canister device (100) for filtering clot material from whole blood, the device (100) comprising:
a. an upper filter chamber (110), configured to receive and contain aspirated whole blood;
wherein the upper filter chamber (110) is at least partially transparent;
b. a lower filter chamber (120) removably attached and fluidly coupled to the upper filter chamber (110), configured to receive and contain filtered whole blood;
c. a coarse filter mesh (130) disposed within the upper filter chamber (110), configured to capture the clot material from the aspirated whole blood;
d. a fine filter mesh (135) disposed within the lower filter chamber (120), configured to filter the aspirated whole blood into filtered whole blood;
e. an inlet port (140) fluidly coupled to the upper filter chamber (110), configured to accept the aspirated whole blood and direct the aspirated whole blood into the upper filter chamber (110); and
f. an outlet port (150) fluidly coupled to the lower filter chamber (120), configured to accept the filtered whole blood from the lower filter chamber (120) and direct the filtered whole blood to an external source;
wherein the device (100) comprises only two filter mesh layers.

2. The device (100) of claim 1, wherein the inlet port (140) comprises a stopcock valve.

3. The device (100) of claim 1, wherein the outlet port (150) comprises a stopcock valve.

4. The device (100) of claim 1, wherein the upper filter chamber (110) and the lower filter chamber (120) are non-vented.

5. The device (100) of claim 1 further comprising one or more air vents fluidly coupled to the upper filter chamber (110).

6. The device (100) of claim 5, wherein the one or more air vents comprise one or more float vents, each float vent (160) comprising:
a. an air port (162) fluidly coupled to air surrounding the device (100) such that air flow is provided in the upper filter chamber (110); and
b. a buoyant component (164) disposed in-line with the air port (162), configured to rise with a fluid level within the upper filter chamber (110) such that the buoyant component (164) blocks the air port (162) at a maximum fluid level in the upper filter chamber (110).

7. The device (100) of claim 5, wherein the one or more air vents comprise one or more check valves, each check valve (170) comprising:
a. an air port (172) fluidly coupled to air surrounding the device (100) such that air flow is provided in the upper filter chamber (110);
b. a spring (174) disposed in-line with the air port (172), configured to move from an extended configuration to a compressed configuration when pressure is applied and move from the compressed configuration to the extended configuration when the pressure is released; and
c. a blocking component (176) coupled to the spring (174) such that the blocking component (176) is configured to block the air port (172) when the spring (174) is in the extended configuration, and the blocking component (176) is configured to release from the air port (172) when the spring (174) is in the compressed configuration such that air flow is permitted through the air port (172);
wherein the aspirated whole blood, upon being directed into the upper filter chamber (110), generates the pressure in the upper filter chamber (110) such that the pressure is applied to the spring (174);
wherein the filtered whole blood, upon being directed out of the lower filter chamber (120), releases the pressure in the upper filter chamber (110) such the pressure is released from the spring (174).

8. The device (100) of claim 1 further comprising a plurality of base legs disposed on the lower filter chamber (120), wherein the plurality of base legs is configured to support the device (100) on a leg of a patient.

9. The device (100) of claim 1, wherein the inlet port (140) is configured to accept a syringe, a catheter, or a combination thereof.

10. The device (100) of claim 1, wherein the outlet port (150) is configured to accept a syringe, a catheter, or a combination thereof.

11. A filter canister device (100) for filtering clot material from whole blood, the device (100) comprising:
a. an upper filter chamber (110), configured to receive and contain aspirated whole blood;
wherein the upper filter chamber (110) is at least partially transparent;
b. a lower filter chamber (120) removably attached and fluidly coupled to the upper filter chamber (110), configured to receive and contain filtered whole blood;
c. a coarse filter mesh (130) disposed within the upper filter chamber (110), configured to capture the clot material from the aspirated whole blood;
d. a fine filter mesh (135) disposed within the lower filter chamber (120), configured to filter the aspirated whole blood into filtered whole blood;
e. an inlet port (140) fluidly coupled to the upper filter chamber (110), configured to accept the aspirated whole blood and direct the aspirated whole blood into the upper filter chamber (110);
f. an outlet port (150) fluidly coupled to the lower filter chamber (120), configured to accept the filtered whole blood from the lower filter chamber (120) and direct the filtered whole blood to an external source;
g. one or more float vents fluidly coupled to the upper filter chamber (110), each float vent (160) comprising:
i. an air port (162) fluidly coupled to air surrounding the device (100) such that air flow is provided in the upper filter chamber (110); and
ii. a buoyant component (164) disposed in-line with the air port (162), configured to rise with a fluid level within the upper filter chamber (110) such that the buoyant component (164) blocks the air port (162) at a maximum fluid level in the upper filter chamber (110); and
h. one or more check valves fluidly coupled to the upper filter chamber (110), each check valve (170) comprising:
i. a second air port (172) fluidly coupled to air surrounding the device (100) such that air flow is provided in the upper filter chamber (110);
ii. a spring (174) disposed in-line with the air port (172), configured to move from an extended configuration to a compressed configuration when pressure is applied and move from the compressed configuration to the extended configuration when the pressure is released; and
iii. a blocking component (176) coupled to the spring (174) such that the blocking component (176) is configured to block the air port (172) when the spring (174) is in the extended configuration, and the blocking component (176) is configured to release from the air port (172) when the spring (174) is in the compressed configuration such that air flow is permitted through the air port (172);
wherein the aspirated whole blood, upon being directed into the upper filter chamber (110), generates the pressure in the upper filter chamber (110) such that the pressure is applied to the spring (174);
wherein the filtered whole blood, upon being directed out of the lower filter chamber (120), releases the pressure in the upper filter chamber (110) such the pressure is released from the spring (174);
wherein the device (100) comprises only two filter mesh layers.

12. The device (100) of claim 11 further comprising a plurality of base legs disposed on the lower filter chamber (120), wherein the plurality of base legs is configured to support the device (100) on a leg of a patient.

13. The device (100) of claim 11, wherein the inlet port (140) is configured to accept a syringe, a catheter, or a combination thereof.

14. The device (100) of claim 11, wherein the outlet port (150) is configured to accept a syringe, a catheter, or a combination thereof.

15. The device (100) of claim 11, wherein the inlet port (140) comprises a stopcock valve.

16. The device (100) of claim 11, wherein the outlet port (150) comprises a stopcock valve.

17. A method for filtering clot material from whole blood, the method comprising:
a. aspirating whole blood from a patient;
b. directing the aspirated whole blood through an inlet port (140) into an upper filter chamber (110) of a filter canister device (100);
c. directing the aspirated whole blood through a coarse filter mesh (130) disposed in the upper filter chamber (110) such that the coarse filter mesh (130) captures the clot material from the aspirated whole blood;
d. visualizing the clot material as captured by the coarse filter mesh (130);
e. directing the aspirated whole blood into a lower filter chamber (120) of the device (100), removably and fluidly coupled to the upper filter chamber (110);
f. directing the aspirated whole blood through a fine filter mesh (135) disposed within the lower filter chamber (120), resulting in filtered whole blood; and
g. directing the filtered whole blood to an external source;
wherein the device (100) comprises only two filter mesh layers.

18. The method of claim 17, wherein the device (100) comprises a plurality of base legs disposed on the lower filter chamber (120), wherein the method further comprises supporting the device (100) on a leg of the patient.

19. The method of claim 17, wherein the inlet port (140) is configured to accept a syringe, a catheter, or a combination thereof.

20. A method for preventing foam buildup while filtering clot material from whole blood, the method comprising:
a. aspirating whole blood from a patient;
b. directing the aspirated whole blood through an inlet port (140) into an upper filter chamber (110) of a vented, non-pressurized filter canister device (100);
c. directing the aspirated whole blood through a coarse filter mesh (130) disposed in the upper filter chamber (110) such that the coarse filter mesh (130) captures the clot material from the aspirated whole blood;
d. visualizing the clot material as captured by the coarse filter mesh (130);
e. directing the aspirated whole blood into a lower filter chamber (120) of the device (100), removably and fluidly coupled to the upper filter chamber (110);
f. directing the aspirated whole blood through a fine filter mesh (135) disposed within the lower filter chamber (120), resulting in filtered whole blood; and
g. directing the filtered whole blood to an external source;
wherein the device (100) comprises only two filter mesh layers.
